(19) United States
(12) Patent Application Publication
(10) Pub. No.: US 2005/0136011 A1
(43) Pub. Date: Jun. 23, 2005

(54) Mousse product and method for conditioning hair

(75) Inventors: Natalia Nekludoff, Lisle, IL (US); Keith Leslie Rutherford, Palatine, IL (US); Liang-Bin Chen, Hoffman Estates, IL (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/946,829

(22) Filed: Sep. 22, 2004

Related U.S. Application Data
(60) Provisional application No. 60/532,242, filed on Dec. 23, 2003.

(51) Int. Cl. A61K 9/00; A61K 7/06; A61K 7/11

(52) U.S. Cl. 424/47; 424/70.12

(57) ABSTRACT

A hair conditioning foamed mousse product is provided which is housed in a pressurized canister fitted with valve having total orifice of at least 0.0002 in² (0.13 mm²). The canister is filled with a cationic surfactant, a high molecular weight silicone, a low molecular weight silicone and a propellant. Advantageously the ratio of low to high molecular weight silicone ranges from 10:1 to 1:10. The low molecular weight silicone in amounts less than 1% counters the anti-foaming effect of the high molecular weight silicone thereby allowing formation of a stable light/airy foam. Also provided is a method for conditioning hair with mousse product applied to hair within a shower stall.

### BACKGROUND OF THE INVENTION

1. Field of the Invention

The invention concerns a conditioner product in mousse form and a method for conditioning hair utilizing this product.

2. The Related Art

The principal reason for conditioning hair is to improve the combability of damaged hair when wet. Other desirable properties may also be imparted to the dry hair such as manageability, softness, body, shine and static control. Also it is expected that a satisfactory conditioner will spread through the hair easily and leave the dry hair feeling clean and not greasy.

Conventional conditioners generally utilize a water soluble cationic surfactant. These formulations may also contain water insoluble conditioners such as non-volatile silicones and/or di-long chain fatty quaternary ammonium compounds.

U.S. Pat. No. 4,859,456 (Marschner) is typical of the hair rinse conditioner technology. Besides cationic surfactant, this reference discloses pyrrolidone copolymers in combination with nonionic cellulose polymers to achieve superior dry feel and lustre. The formulas may further contain silicones such as dimethiconols and can be delivered with propellant in mousse form.

U.S. Pat. No. 6,613,316 B2 (Sun et al.) discloses aqueous opaque hair conditioners based on a combination of monoalkyl quat (cationic surfactant) and a mixture of C16, C16 dialkyl quat and C18, C18 dialkyl quat. Optionally, a silicone compound may be incorporated into the formula. Typical silicones were said to include amodimethicone, dimethicone, dimethiconol and decamethylcyclopentasiloxane (D5).

U.S. Pat. No. 6,290,932 B2 (Pratley et al.) reports aerosol-delivered hair styling aids. These may contain a quaternary ammonium compound, volatile silicone and non-volatile silicone.

Commercially successful mousse type hair conditioners require the product to have a high quality foam. Consumers correlate foam quality with the conditioning and volume that the product will impart to the hair. Furthermore, it is important to tune both initial creaminess and rate of foam collapse. These properties relates to the ease which a consumer can dispense product from an aerosol container. Moreover, these properties when carefully tuned provide the pleasurable handle and quick dispersibility into wet hair desired by consumers.

Non-volatile silicones such as dimethicone are generally necessary to impart good conditioning, especially for damaged hair. Unfortunately the presence of non-volatile silicones has significant deleterious effect on initial and lifetime quality of the foam. Even small amounts of non-volatile silicone can impart a powerful anti-foaming effect. Collapse can occur within seconds.

### SUMMARY OF THE INVENTION

A hair conditioning mousse product is provided which includes:
(i) a canister fitted with a spray nozzle and a valve of total orifice size greater than 0.0002 in² (0.13 mm²); and
(ii) a hair conditioning composition held within the canister and dispensible therefrom, the composition including:
   (a) from about 0.01 to about 10% of a cationic surfactant by weight of the composition;
   (b) from about 0.001 to about 2% of a high molecular weight silicone by weight of the composition;
   (c) from about 0.001 to less than 1% of a low molecular weight silicone by weight of the composition;
   (d) from about 0.5 to about 10% of a propellant by weight of the composition; and
      wherein the low molecular weight to high molecular weight silicones are present in a weight ratio ranging from about 10:1 to about 1:10.

Furthermore, a method is provided for conditioning hair which includes:
(A) providing a hair conditioning mousse product which includes:
   (i) a canister fitted with a spray nozzle and a valve of total orifice size greater than 0.0002 in² (0.13 mm²); and
   (ii) a hair conditioning composition held within the canister and dispensible therefrom, the composition including:
   (a) from about 0.01 to about 10% of a cationic surfactant by weight of the composition;
   (b) from about 0.001 to about 2% of a high molecular weight silicone by weight of the composition;
   (c) from about 0.001 to less than 1% of a low molecular silicone by weight of the composition;
   (d) from about 0.5 to about 10% of a propellant by weight of the composition; and
      wherein the low molecular weight to high molecular weight silicones are present in a weight ratio ranging from about 10:1 to about 1:10;
(B) placing a person intending to use the product in a shower stall having an overhead fixture for delivery of a water spray;
(C) dispensing foam mousse from the product and placing the foam mousse onto hair of the person; and
(D) rinsing with water from the fixture the foam mousse placed onto the hair.

### DETAILED DESCRIPTION OF THE INVENTION

Now it has been found that anti-foam effects of high molecular weight silicone conditioner can be overcome by formulating with a relatively small amount of a low molecular weight silicone. Incorporation of less than 1% of decamethylcyclopentasiloxane (D5) has been found to systematically offset the foam deterioration caused by dimethiconol or other high molecular weight silicones. It has also been found advantageous although not limiting to formulate with a weight ratio of low to high molecular weight silicone ranging from about 10:1 to about 1:10. Still further, it is advantageous for the dispensing canister to have a valve with total orifice area greater than 0.0002 in² (0.13 mm²). Spray rates, evacuation and ease of dispensing are poor for small orifice dispensing areas.

Cationic surfactants of the present invention are generally water-soluble quaternary ammonium compounds having one or two long chain alkyl groups containing from about 8 to about 22 carbon atoms. The long chain alkyl groups also can include, in addition to, or in replacement of, carbon and hydrogen atoms, ether linkages or similar water-solubilizing linkages. The remaining two or three substituents of the quaternary nitrogen of the quaternary ammonium compound can be hydrogen; or benzyl; or short chain alkyl or hydroxyalkyl groups, such as methyl, ethyl, hydroxymethyl or hydroxyethyl groups; or combinations thereof, either of the same or of different identity, as long as the quaternary ammonium compound is water soluble. Therefore, the water-soluble quaternary ammonium compound can be depicted by the following general structural formula: 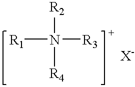
wherein R₁ is an alkyl group including from about 9 to about 22 carbon atoms; R₂ is selected from the group consisting of an alkyl group including from about 8 to about 22 carbon atoms, a hydrogen atom, a methyl group, an ethyl group, a hydroxymethyl group and a hydroxyethyl group; R₃ and R₄ is selected from the group consisting of a hydrogen atom, a methyl group, an ethyl group, a hydroxymethyl group and a hydroxyethyl group; and X is a water soluble anion non-limiting examples of which are chloride, methosulfate, ethosulfate bromide, tosylate, acetate, phosphate and nitrate anions. However, it should be noted that the quaternary nitrogen of the wter-soluble quaternary ammonium compound also can be included in a heterocyclic nitrogen-containing moiety, such as morpholine or pyridine.

Cationic surfactants are hereby further defined in the alternative as materials that, when mixed with water, form a true solution such that the quaternary ammonium compound, when present up to its saturation point, will not separate from the water phase. Consequently, the following water-soluble quaternary ammonium compounds are exemplary, but not limiting, of water-soluble quaternary ammonium compounds that can be used in the method and composition of the present invention:

| | |
|---|---|
| Lauryltrimethylammonium chloride | (Laurtrimonium chloride); |
| Stearyltri(2-hydroxyethyl) ammonium | (Quaternium-16); |
| chloride | |
| Lauryldimethylbenzylammonium | (Lauralkonium chloride); |
| chloride | |
| Oleyldimethylbenzylammonium | (Olealkonium chloride); |
| chloride | |
| Dilauryldimethylammonium | (Dilauryldimonium chloride); |
| chloride | |
| Cetyldimethylbenzylammonium | (Cetalkonium chloride); |
| chloride | |
| Dicetyldimethylammonium | (Dicetyldimonium chloride); |
| chloride | |
| Laurylpyridinium chloride | (Laurylpyridinium chloride) |
| Cetylpyridinium chloride | (Cetylpyridinium chloride); |
| N-(soya alkyl)-N,N,N-trimethyl | (Soyatrimonium chloride) |
| ammonium chloride | |
| Polydiallyldimethylammonium chloride | (Polyquaternium-6); |
| Diallyldimethyl ammonium salt | (Polyquaternium-7); |
| copolymerized with acrylamide | |
| Guarhydroxypropyltrimonium chloride | (Guarhydroxypropyl- |
| | Trimonium chloride); |
| Copolymer of N-vinyl-pyrrolidone | (Polyquaternium-11); |
| and N,N-dimethylaminoethylmethacrylate, | |
| quaternized with dimethyl-sulfate | |
| Copolymer of acrylamide and N,N- | (Polyquaternium-5); |
| dimethylamino-ethyl methacrylate, | |
| quaternized with dimethyl sulfate | |
| Cationic hydroxyethylcellulosics | (Polyquaternium-10); |
| Cetyltrimethylammonium chloride | (Cetrimonium chloride); |
| Decyldimethyloctylammonium chloride | (Quaternium-24); |
| Myristyltrimethylammonium chloride | (Mytrimonium chloride); |
| Polyoxyethylene (2)-cocomonium | (PEG-2 Cocomonium |
| chloride | chloride); |
| Methylbis(2-hydroxyethyl) | (PEG-2 Cocoyl Quaternium- |
| Cocoammonium chloride | 4); |
| Methylpolyoxyethylene (15) | (PEG-15 Cocoyl Quaternium- |
| Cocoammonium chloride | 4); |
| Methylbis(2-hydroxyethyl) | (PEG-2 Stearyl Quaternium- |
| octadecyl ammonium chloride | 4); |
| Methylpolyoxyethylene- | (PEG-15 Stearyl |
| (15) octadecylammonium chloride | Quaternium-4); |
| Methylbis(2-hydroxyethyl)- | (PEG-2 Oleyl |
| Oleylammonium chloride | Quaternium-4); |
| Methylpolyoxyethylene-(15) | (PEG-15 Oleyl |
| Oleylammonium chloride | quaternium-4); |

Whereinabove the name in parenthesis is the compound name given by the Cosmetic, Toiletry and Fragrance Association, Inc. in the CTFA Cosmetic Ingredient Dictionary, 3^{rd} ed., 1982, hereinafter referred to as the CTFA Dictionary.

It should be noted that a long alkyl chain of the water-soluble quaternary ammonium compound does not have to be solely, or primarily, of one chain length, i.e, the long chain need not be only lauryl (C₁₂) or myristyl (C₁₄). Rather, a quaternary ammonium compound wherein the long alkyl chain is a mixture of lengths can be used, as long as the quaternary ammonium compound is water soluble. Such conditioning agents are prepared conveniently from naturally-occurring materials, such as tallow, coconut oil, soya oil and the like, or from synthetically produced mixtures Examples of water-soluble quaternary ammonium compounds having mixed carbon chain lengths include N-(soyaalkyl)-N,N,N-trimethyl ammonium chloride (soyatrimonium chloride) and polyoxyethylene-2-cocomonium chloride (PEG-2 cocomonium chloride).

Amounts of the cationic surfactant may range from about 0.01 to about 10%, preferably from about 0.1 to about 5%, more preferably from about 0.5 to about 2% and optimally from about 0.7 to about 1% by weight of the composition.

According to some embodiments of the present invention, the composition may also include an oil-soluble, water-dispersible quaternary ammonium compound.

An oil-soluble, water-dispersible quaternary ammonium compound useful in the composition is a quaternary ammonium compound having one or two long chain alkyl groups including from about 14 to about 22 carbon atoms. The remaining two to three substituents present on the quaternary nitrogen of the quaternary ammonium compound can be hydrogen; or benzyl; or short chain alkyl groups, such as methyl, or ethyl; or combinations thereof, as long as the quaternary ammonium compound is oil soluble and water dispersible. Therefore, the oil-soluble quaternary ammonium compound can be depicted by the following general structural formula: 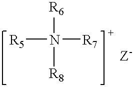
wherein R₅ is an alkyl group including from about 14 to about 22 carbon atoms; R₆ is selected from the group consisting of an alkyl radical including from about 14 to 22 carbon atoms, a methyl radical and an ethyl radical; R₇ is selected from the group consisting of a benzyl, a methyl and an ethyl radical; R₈ is selected from the group consisting of a methyl and an ethyl radical; and Z⁻ is selected from the group consisting of chloride, bromide, methosulfate, ethosulfate, tosylate, acetate, nitrate and phosphate. However, it should be noted that the quaternary nitrogen of the oil-soluble quaternary ammonium compound can be included in a heterocyclic nitrogen-containing moiety such as pyridine or morpholine.

The anion of the oil-soluble quaternary ammonium compound can be any common anion as long as the quaternary ammonium compound is oil soluble. It should be noted that, in certain instances, it is the anionic portion of the quaternary ammonium compound that determines whether the quaternary ammonium compound is water soluble or oil soluble. For example, in comparing the quaternary ammonium compounds cetyltrimethylammonium chloride (cetrimonium chloride), cetyltrimethylammonium bromide (cetrimonium bromide) and cetyltrimethylammonium p-toluenesulfonate (cetrimonium tosylate), the cations of the quaternary ammonium compounds are identical. However, cetrimonium chloride is water soluble, whereas cetrimonium bromide and cetrimonium tosylate are oil soluble. Therefore, a change in identity of the anion can effectively change the solubility characteristics of the quaternary ammonium compound.

In additon, other seemingly minor variations in molecular structure can significantly effect the solubility characteristics of a quaternary ammonium compound. For example, dramatic effects are demonstrated by varying the carbon chain length of the long alkyl chain of the quaternary ammonium compound. In general, the water solubility of the quaternary ammonium compound decreases as the carbon chain length of the long alkyl chain of a quaternary ammonium compound increases. Consequently, cetyidimethylbenzylammonium chloride (cetalkonium chloride) is water soluble, whereas the addition of two carbon atoms renders the resulting stearyidimethylbenzylammonium chloride (stearalkonium chloride) water insoluble and oil soluble.

An oil-soluble, water-dispersible quaternary ammonium compound is hereby further defined as a compound that when mixed with a non-polar solvent, like a hydrocarbon, forms a true solution, such that the compound, when present up to its saturation point, will not separate from the oil phase; and that, when mixed with water, is dispersed when stirred or agitated, but separates from the water phase when stirring or agitation is stopped. Therefore, the following list of oil-soluble quaternary ammonium compounds are exemplary, but not limiting, of oil-soluble, water-dispersible quaternary ammonium compounds that can be used in the method and composition of the present invention:

| | |
|---|---|
| Cetyldimethylethylammonium bromide | (Cetethyldimonium bromide); |
| Cetyltrimethylammonium | (Cetrimonium tosylate); |
| p-toluenesulfonate | |
| Stearyldimethylbenzylammonium | (Stearalkonium chloride); |
| Chloride | |
| Distearyldimethylammonium chloride | (Distearyldimonium chloride); |
| Dimethyldi(hydrogenated tallow) | (Quaternium-18); |
| ammonium chloride | |
| Cetyltrimethylammonium bromide | (Cetrimonium bromide); |
| Cetylethylmorpholinium ethosulfate | (Cetethylmorpholinium |
| | ethosulfate); |
| Behenyldimethylbenzylammonium | (Behenalkonium chloride); |
| chloride | |
| Behenyltrimethylammonium chloride | (Behentrimonium chloride); |
| Myristyltrimethylammonium bromide) | (Mytrimonium bromide); |

wherein the name in the parenthesis is the compound name given in the CTFA Dictionary.

Most preferred are embodiments incorporating an oil-soluble, water-dispersible conditioning agent such as a dialkyl quat mixture of di-C₁₈ alkyl quaternary ammonium compound and di-C₆ alkyl quaternary ammonium compound, the mixture being present in a weight ratio of about 1:5 to about 5:1, preferably from about 1:2 to about 2:1 by weight. Illustrative is Quaternium-18 which is a dimethyl di(hydrogenated tallow) ammonium chloride.

Oil soluble, water-dispersible conditioning agents may range from about 0.01 to about 5%, preferably from about 0.1 to about 2%, optimally from about 0.2 to about 1% by weight of the composition.

High molecular weight silicones will be present in compositions of this invention. These are characterized as having a number average molecular weight higher than 6,000, preferably, higher than 10,000, more preferably higher than 100,000, even more preferably higher than 500,000. These silicones may be selected from but are not necessarily exclusive to polydialkylsiloxanes, polydiarylsiloxanes and polyalkarylsiloxanes. The polyalkylsiloxanes correspond to the general chemical formula R₃SiO[SiO]SiR₃ wherein R is an alkyl group (preferably R is methyl or ethyl, more preferably methyl) and x is an integer up to about 500, chosen to achieve the desired molecular weight. Commercially available polyalkylsiloxanes include the polydimethylsiloxanes, which are also known as dimethicones, nonlimiting examples of which include the Vicasil® series sold by General Electric Company and the Dow Corning® 200 series sold by Dow Corning Corporation. Specific examples of polydimethylsiloxanes useful herein include Dow Corning® 200 fluid having a viscosity of 10,000 centistokes (1×10⁻² m²/s) and a boiling point greater than 250° C.

Also useful are materials such as trimethylsiloxysilicate, which is a polymeric material corresponding to the general chemical formula [(CH₂)₃SiO_{1/2}]ₓ[SiO₂]_{y} wherein x is an integer from about 1 to about 500 and y is an integer from about 1 to about 500. A commercially available trimethylsiloxysilicate is sold by Dow Corning as a mixture with dimethicone.

Another useful type of high molecular weight silicone are dimethiconols, which are hydroxy terminated dimethyl silicones. These materials can be represented by the general chemical formulas R₃SiO[R₂SiO]ₓSiR₂OH and HOR₂SiO[R₂SiO]ₓSiR₂OH wherein R is an alkyl group (preferably R is methyl or ethyl, more preferably methyl) and x is an integer up to about 500, chosen to achieve the desired molecular weight. Often the dimethiconoles silicones are commercially available as pre-formed emulsions in water. Suitable commercially available emulsified dimethiconols are DC 1784, DC 1785, DC 1786 and DC 929.

Still another suitable type of high molecular weight silicone are alkyl modified siloxanes such as alkyl methicones and alkyl dimethicones wherein the alkyl chain contains 10 to 50 carbons. Such siloxanes are commercially available under the tradenames ABIL WAX 9810 (C₂₄-C₂₈ alkyl methicone) (sold by Goldschmidt) and SF1632 (cetearyl methicone) (sold by General Electric Company). As a general rule, high molecular weight silicones are polysiloxanes having boiling points greater than 250° C. at atmospheric pressure. They also can have a viscosity ranging from 120 cst to at least 1 million cst (1.2×10⁻⁴ to at least 1 m²/s).

Amounts of the high molecular weight silicones may range from about 0.01 to about 2%, preferably from about 0.1 to about 1%, optimally from 0.2 to about 0.5% by weight of the composition.

Low molecular weight silicone compounds of the present invention are organopolysiloxanes having number average molecular weights no higher than 6,000, preferably less than 3,000, more preferably less than 1,500, and optimally less than 800. Advantageously but not necessarily these silicones can have boiling points of less than 250° C., and more preferably less than 200° C. at atmospheric pressure. They also can have a viscosity ranging from about 0.01 to 100 cst (1×10⁻⁸ m²/s to 1×10⁻⁴ m²/s). These materials are usually linear or cyclic polydimethylsiloxanes. An example of a linear, low molecular weight polydimethylsiloxane compound useful in the composition and method of the present invention is hexamethyldisiloxane, available commercially under the trademark Dow Corning 200 Fluid, from Dow Corning Corp., Midland, Mich. Hexamethyidisiloxane has a viscosity of 0.65 cst (0.65×10⁻⁶ m²/s). Other linear polydimethylsiloxanes, such as decamethyltetrasiloxane, having a boiling point of about 195° C. and a viscosity of 1.5 cst (1.5×10⁻⁶ m²/s); octamethyltrisiloxane; and dodecamethylpentasiloxane, also may be used in the compositions of the present invention. In addition, the cyclic, low molecular weight polydimethylsiloxanes, named in the CTFA Dictionary as cyclomethicones, can be used in the composition. The cyclomethicones are water-insoluble cyclic compounds having an average of about 3 to about 6-[O-Si(CH₃)₂]-repeating group units per molecule and boil at atmospheric pressure in a range of from about 150° C. to about 198° C. The polydimethyl cyclosiloxanes having an average of about 4 to about 5 repeating units per molecule, i.e., the tetramer and pentamer, are preferred. Particularly preferred for purposes of the present invention is decamethylcyclopentasiloxane (D5) available commercially as DC 245 and DC 246.

Other useful low molecular weight silicones are polyalkylaryl siloxanes, with polymethylphenyl siloxanes having viscosities from about 15 to about 65 centistokes (15×10⁻⁶ m²/s to 65×10⁻⁶ m²/s) at 25° C. being preferred. These materials are available, for example, as SF 1075 methylphenyl fluid (sold by General Electric Company) and 556 Cosmetic Grade phenyl trimethicone fluid (sold by Dow Corning Corporation). Alkylated silicones such as methyldecyl silicone and methyloctyl silicone are useful herein and are commercially available from General Electric Company.

Amounts of the low molecular weight silicone range from about 0.001 to less than 1%, preferably from about 0.01 to about 0.5, more preferably from about 0.1 to about 0.3, and optimally from 0.15 to 0.25% by weight of the composition.

According to the present invention, the weight ratio of low to high molecular weight silicone can range from about 10:1 to about 1:10, preferably from about 5:1 to about 1:10, more preferably from about 2:1 to about 1:5, optimally from about 1:1 to about 1:3.

Propellants are included in compositions of this invention. The propellant is any liquefiable gas conventionally used for aerosol canisters. Examples include dimethylether, propane, n-butane, isobutane, pentane, isopentane and mixtures thereof. These are, for example, available commercially as A17, A46 and A75 from the Philips Petroleum Company. Halogenated materials may also be used including trichlorofluoromethane, dichlorofluoromethane, dichlorotetrafluoroethane and halocarbon mixtures thereof. Other examples of suitable propellants include nitrogen, carbon dioxide and compressed air. Amounts of the propellant may range from about 0.5 to about 10%, preferably from about 1 to about 50%, optimally from about 2 to about 4% by weight of the composition.

Fatty (C₁₀-C₂₄) alcohols are useful for compositions of the present invention. Non-limiting examples include cetyl alcohol, stearyl alcohol, myristyl alcohol, behenyl alcohol and mixtures thereof. Particularly preferred is cetearyl alcohol which is a mixture of C₁₆ and C₁₈ alkyl alcohols. Amounts of the fatty alcohol may range from about 0.1 to about 1 5%, preferably from about 1 to about 10%, more preferably from about 1.5 to about 5% by weight of the composition.

Water will also be present in the compositions. Amounts may range from 40 to about 97%, preferably from about 16 to about 95%, optimally from about 80 to about 90% by weight of the composition.

Also present may be hydrophilic conditioning agents. Nonlimiting examples include polyhydric alcohols, polypropylene glycols, polyethylene glycols, ethoxylated and/or propoxylated C₃-C₆ diols and triols, ethoxylated and/or propoxylated sugars, sugar alcohols and mixtures thereof. Examples of polyhydric alcohols include glycerin and neopentyl alcohol. Polyethylene glycols are represented by PEG-2, PEG-3, PEG-4 and PEG-50. Illustrative sugars include sucrose, fructose, glucose, sorbitol and mannitol. Other useful glycols include hexylene glycol, butylene glycol, isoprene glycol and 2-methyl-1,3-propanediol (MP® Diol). Most preferred is glycerin.

Amounts of the hydrophilic conditioning agents may range from about 0.01 to about 20%, preferably from about 0.1 to about 10%, more preferably from about 0.5 to about 5%, optimally from about 0.8 to about 1.5% by weight of the composition.

Further optional components may be sunscreen agents. These may either be water soluble or insoluble organic substances. Nonlimiting examples include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyidibenzoyl methane, 4-isopropyl dibenzoyl methane, 3-benzylidene camphor, 3-(4-methylbenzylidene) camphor and mixtures thereof. Amounts of the sunscreen agents when present may range from about 0.000001 to about 5%, preferably from about 0.00001 to about 1%, optimally from 0.00001 to about 0.1% by weight of the composition.

Vitamins may optionally be included. Illustrative are Vitamin A (e.g. beta-carotene, retinoic acid, retinol, retinyl palmitate, retinyl propionate and other retinoids), Vitamin B (e.g. niacin, niacinamide, riboflavin, pantothenic acid and derivatives thereof), Vitamin C (e.g. ascorbic acid, ascorbyl tetraisopalmitate, ascorbyl magnesium phosphate and other ascorbic salts), Vitamin D, Vitamin E (e.g. tocopherol, tocopherol acetate and other ester derivatives) and mixtures thereof. Amounts of the vitamin may range from about 0.000001 to about 1%, preferably from about 0.00001 to about 0.01, optimally from about 0.1 to about 0.5% by weight.

Chelating agents may also be incorporated into the compositions. Particularly preferred are the salts of ethylene diamine tetraacetic acid (EDTA) including tetrasodium EDTA and disodium EDTA. Organophosphorous chelating agents may also be employed. These are commercially available under the trademark Dequest®. Amounts of the chelating agents may range from 0.01 to about 2%, preferably from about 0.1 to about 1% by weight of the composition.

Preservatives are generally important to control microbe growth in aqueous systems. Typical preservatives include but are not limited to phenoxyethanol, methyl paraben, ethyl paraben, propyl paraben, butyl paraben, potassium sorbate, Kathon CG® (a mixture of methylchloroisothiazolinone and methylisothiazolinone), DMDM Hydantoin, iodopropynyl butyl carbamate and mixtures thereof. Amounts of the preservative may range from about 0.000001 to about 1%, preferably from about 0.0001 to about 0.5% by weight of the composition and dependent upon the activity of any particular preservative.

Other adjunct components of compositions according to the present invention may include amino acids and salts thereof (e.g. lysine, arginine, cysteine, tyrosine, glutamine, proline and combinations thereof), fragrances, colorants, anti-corrosion agents, and hair benefit agents (e.g. phytantriol, borage extract, ceramides) and combinations.

Canisters for dispensing the compositions may be formed from metal, plastics and combinations of these materials. Particularly preferred are aluminum canisters. Due to the often high water content of the compositions, presence of quat and chloride ions and a relatively acidic pH, risk of corrosion is high. This problem can be counteracted by lining the aluminum interior surface with an insultating film such as an epoxy, an epoxy phenolic or polyamideimide (PAM) liner. For purposes of this invention it has been found that a PAM liner provided the best resistance against corrosion.

Expression of the product from the pressurized aerosol canister is controlled by a valve. Products passes through a restrictive orifice frequently known. as the stem orifice. For purposes of this invention the total orifice area of the valve should be greater than 0.0002 in² (0.13 mm²), preferably greater than 0.0003 in² (0.19 mm²), more preferably greater than 0.0005 in² (0.32 mm²), and optimally greater than 0.0008 in² (0.52 mm²). Total orifice area means the cumulative area available for product flow as it exits the valve. The term “total surface area” includes the sum of the exit areas on valves with multiple openings.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

A set of experiments were conducted to evaluate the effect of different levels of cyclomethicone (D5) and dimethiconol on foam properties. The test formulas are outlined in Table I. Formula A utilizes cetrimonium chloride as the cationic surfactant and Quaternium-18 as an adjunct oil-soluble type conditioner. Formula B utilizes cetrimonium chloride as the cationic surfactant and is the sole quaternary ammonium component. Formula A and B compositions were placed into aluminum canisters lined with PAM and fitted with dispensing valves having a total orifice are of 0.000981748 in² (0.63 mm²). The pH of the compositions were held within the range 5.5-6.2.

The foam was characterized in a Rheometric ARES Rheometer using a 50 mm parallel plate geometry with 2.5 mm gap spacing at 25° C. This larger than normal gap spacing was necessary because some of the poorer quality foams can be squashed and collapsed pre-maturely with smaller spacings. To improve sensitivity of the larger gap, the 50 mm parallel plate was employed. Dynamic oscillation at a fixed frequency of 10 rad/s is applied to the foam in a linear viscoelastic region for a period of time. The modulus is monitored over a period of time (500 seconds) to assess the initial firmness and the foam decay characteristics. Initial firmness is monitored by the elastic modulus at 10 seconds. Half life of the foam is the time at which the elastic modulus reaches half of its initial value. Half life reflects the breaking time of foam. This is the measure of the time within which initial stiffness of the foam has dropped to half its value.

TABLE I

| | FORMULA A | FORMULA B |
|---|---|---|
| COMPONENT | (weight %) | (weight %) |
| A46 Propellant | 6.0 | 6.0 |
| Cetyl/Stearyl Alcohol | 2.8 | 2.8 |
| Cetrimonium Chloride (30% Active) | 2.4 | 3.3 |
| Glycerin | 0.9 | 0.9 |
| Fragrance | 0.6 | 0.6 |
| Quaternium-18 & PG | 0.4 | — |
| Preservatives | 0.2 | 0.2 |
| DC 1785 (60% Dimethiconol | * | * |
| Emulsion) | | |
| Decamethylcyclopentasiloxane (D5) | * | * |
| Deionized Water | To 100 | To 100 |

* Amounts found in Table II

Table II summarizes foam properties for different ratios and levels of D5 and dimethiconol within Formula A. Values for Elastic Modulus within the range of 0 to 1 50 Pa reflect foam that is too fluffy/airy thereby being undesirable. Values greater than 400 Pa reflect foam that is relatively too solid/stiff, becomes lumpy (poor texture) and thereby also undesirable. Within the range of 150 to 400 Pa the Elastic Modulus reflects a foam aesthetically pleasing to consumers.

TABLE II
Formula A Performance

| | | Level of | | G′ (Pa) @ 10 Sec | | Visual |
|---|---|---|---|---|---|---|
| | Level of D5 | Dimethiconol | Ratio | (Elastic Modulus | Half Life | Appearance after |
| Experiment No. | (weight %) | (weight %) | D5:Dimethiconol | of Foam) | (seconds) | 30 sec |
| 1 | 0 | 0 | n/a | 310 | 43 | Rich & |
| | | | | | | creamy |
| 2 | 1 | 0 | 0 | 449 | 69 | Very rich |
| | | | | | | & creamy |
| 3 | 1 | 0.01 | 100:1 | 430 | 73 | Very rich |
| | | | | | | & creamy |
| 4 | 1 | 1 | 1:1 | 367 | 81 | Stable |
| | | | | | | creamy |
| 5 | 1 | 2 | 1:2 | 326 | 88 | Stable |
| | | | | | | creamy |
| 6 | 0 | 2 | 0 | 124 | 38 | Poor |
| | | | | | | untable |
| 7 | 0.05 | 2 | 1:40 | 129 | 37 | Poor |
| | | | | | | unstable |
| 8 | 0.2 | 2 | 1:10 | 178 | 36 | More |
| | | | | | | stable light/airy |
| 9 | 1 | 0.1 | 10:1 | 360 | 79 | Rich & creamy |

TABLE III
Formula B Performance

| | | Level of | | G′ (Pa) @ 10 Sec | | Visual |
|---|---|---|---|---|---|---|
| | Level of D5 | Dimethiconol | Ratio | (Elastic Modulus | Half Life | Appearance after |
| Experiment No. | (weight %) | (weight %) | D5:Dimethiconol | of Foam) | (seconds) | 30 sec |
| 10 | 1 | 0 | Infinity | 489 | 68 | Very Rich & |
| | | | | | | creamy |
| 11 | 1 | 0.01 | 100:1 | 486 | 66 | Very rich |
| | | | | | | & creamy |
| 12 | 1 | 1 | 1:1 | 365 | 41 | Very rich |
| | | | | | | & creamy |
| 13 | 1 | 2 | 1:2 | 206 | 38 | Rich & |
| | | | | | | creamy |
| 14 | 0 | 2 | 0 | 121 | 38 | Poor |
| | | | | | | Creamy |
| 15 | 0.05 | 2 | 1:40 | 90 | 23 | Poor |
| | | | | | | Unstable |
| 16 | 0.2 | 2 | 1:10 | 94 | 28 | Poor |
| | | | | | | Unstable |

Low levels of decamethylcyclopentasiloxane (D5) offset foam deterioration resulting from the presence of dimethiconol. However, for favorable results not only must D5 be at low levels but in particular ratios to the dimethiconol. If the level or ratio is too low, then the foam is not stabilized. If the level or ratio is too high, the foam becomes solid in appearance and does not distribute well through hair.

Experiment 3 foam exhibits a much too high Elastic Modulus (430 Pa) resulting from a very high D5:Dimethiconol ratio of 100:1. Experiment 7 at the other extreme (Elastic Modulus of 129 Pa) has a ratio of 1:40 resulting in a foam of poor visual appearance and instability. By contrast, Experiment 8 with a ratio of 1:10 has satisfactory stability and a still light/airy foam. Similar results can be seen from the Experiments in Table III.

### EXAMPLE 2

Effects of small amounts of D5 were evaluated in a large scale consumer test. There were 186 participants. Each participant evaluated two formulas. These were essentially identical to the base Formula A shown in Table I of the first Example. The first test formula (I) incorporated 0.3% (active basis) dimethiconol but no D5. The second formula (II) besides 0.3% dimethiconol was charged with 0.1 5% D5. The participants were requested to rate the following attributes:
PERFORMANCE ATTRIBUTES
More Manageable
More Volume/fullness
More Root Lift/Poofiness
More Body
Less Buildup
Style Lasts Longer

Formula II was statistically favored (95% confidence level) for all of the above listed performance attributes. Again this test confirmed that a small amount of D5 provided unexpected performance advantages.

### EXAMPLE 3

This Example reports a study to determine optimum total orifice size. A formula similar to that reported in Table I was charged into a pressurized aluminum can lined with PAM to resist corrosion. Two different valves were evaluated. The first had a total orifice area of 0.000981748 in² (0.63 mm²). The second had a total orifice area of 0.000132732 in² (0.086 mm²). Poor results were found with respect to the latter in the clinical properties of spray rate, evacuation and ease of dispensing. By contrast, the valve with total surface area of 0.000981748 in² (0.63 mm²) had very satisfactory performance for all of the dispensing criteria.

### EXAMPLE 4

The effects of D5 concentrations were evaluated with respect to foam and conditioning properites. Dimethiconol level was held fixed at 0.31% for these experiments. Table IV outlines the results.

TABLE IV

| | | G′(Pa) @ 500 Sec (Elastic |
|---|---|---|
| Experiment No. | Level of D5 (Weight %) | Modulus of Foam) |
| 17 | 0 | 0 |
| 18 | 0.025 | 10 |
| 19 | 0.05 | 120 |
| 20 | 0.1 | 280 |
| 21 | 0.2 | 380 |
| 22 | 0.4 | 390 |
| 23 | 0.8 | 385 |
| 24 | 1.0 | 375 |
| 25 | 5.0 | 280 |

Opitimum results with D5 in the specified system is achieved at concentrations between about 0.2 and about 1% by weight D5.

1. The hair conditioning mousse product comprising:
(i) a canister fitted with a spray nozzle and a valve of total orifice size greater than 0.0002 in² (0.13 mm²); and
(ii) a hair conditioning composition held within the canister and dispensible therefrom, the composition comprising:
(a) from about 0.01 to about 10% of a cationic surfactant by weight of the composition;
(b) from about 0.001 to about 2% of a high molecular weight silicone by weight of the composition;
(c) from about 0.001 to less than 1% of a low molecular silicone by weight of the composition;
(d) from about 0.5 to about 10% of a propellant by weight of the composition; and
wherein the low molecular weight to high molecular weight silicones are present in a weight ratio ranging from about 10:1 to about 1:10;

2. The product according to claim 1 wherein the low molecular weight silicone has a viscosity that is no higher than 100 cst (1×10⁻⁴ m²/s) and is selected from cyclomethicone, phenyltrimethicone and linear dimethicone.

3. The product according to claim 1 wherein the low molecular weight silicone is decamethyl cyclopentasiloxane.

4. The product according to claim 1 wherein the low molecular weight silicone has a viscosity ranging from 0.65 to 100 cst (0.65×10⁻⁶ to 1×10⁻⁴ m²/s).

5. The article according to claim 1 wherein the ratio of low to high molecular weight silicone ranges from about 2:1 to 1:5.

6. The article according to claim 1 wherein the low molecular weight silicone is present at a concentration from about 0.05 to about 0.5% by weight of the composition.

7. The article according to claim 1 further comprising an oil soluble quaternary ammonium compound which is a mixture of a di-C₁₆ alkyl and di-C₁₈ alkyl quat present in a relative ratio of 5:1 to 1:5 and also in an amount from about 0.01 to about 5% by weight of the composition.

8. A method for conditioning hair comprising:
(A) providing a hair conditioning product comprising:
(i) a canister fitted with a spray nozzle and a valve of total orifice size greater than 0.0002 in² (0.13 mm²); and
(ii) a hair conditioning composition held within the canister and dispensible therefrom, the composition including:
(a) from about 0.01 to about 10% of a cationic surfactant by weight of the composition;
(b) from about 0.001 to about 2% of a high molecular weight silicone by weight of the composition;
(c) from about 0.001 to less than 1% of a low molecular silicone by weight of the composition;
(d) from about 0.5 to about 10% of a propellant by weight of the composition; and
wherein the low molecular weight to high molecular weight silicones are present in a weight ratio ranging from about 10:1 to about 1:10;
(B) placing a person intending to use the product in a shower stall having an overhead fixture for delivery of a water spray;
(C) dispensing foam mousse from the product and placing the foam mousse onto hair of the person; and
(D) rinsing with water from the fixture the foam mousse placed onto the hair.
